(19) United States
(12) Patent Application Publication
(10) Pub. No.: US 2011/0051944 A1
(43) Pub. Date: Mar. 3, 2011

(54) Laryngophone combined with transmitter and power source

(76) Inventor: Lois Margaret Kirkpatrick, Centreville, VA (US)

(21) Appl. No.: 12/807,181

(22) Filed: Aug. 30, 2010

Related U.S. Application Data
(60) Provisional application No. 61/275,560, filed on Sep. 1, 2009.

(51) Int. Cl. A61F 2/20

(52) U.S. Cl. 381/70

(57) ABSTRACT

A method of transmitting the human voice comprising an apparatus worn around the user's neck that contains a laryngophone (“throat microphone”), a transmitter and a power source.

### CROSS REFERENCE TO RELATED APPLICATIONS

This application is related to provisional patent granted to applicant: U.S. 61/275,560.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND OF THE INVENTION

### Field of the Invention

The present invention is in the field of wireless microphones and transmitters worn by the user. More particularly, the present invention is in the field of contact microphones known as laryngophones or “throat mics.”

To use a conventional laryngophone (“throat microphones”), the user must have an additional device which is the transmitter and power source located inside a container that is separate from the microphone. The transmitter enables sound to be transmitted from the microphone to a receiver or sound amplification system that is separate and at some distance from the user. Users must carry or wear the transmitter/power source unit in addition to wearing the layrngophone.

Having to wear or carry the transmitter and power source in a separate unit can be cumbersome and distracting, especially when the user is engaged in physical activity, including the performing arts, sports, emergency response operations, law enforcement or combat. In such cases, the separate transmitter/power source unit can inhibit the user's physical movements.

In the performing arts arena, the separate transmitter/power source unit can be aesthetically unappealing, and must be hidden under costumes or clothing. The separate transmitter/power source unit can be challenging to public speakers wearing outfits that do not have anything to which the user can attach the separate transmitter/power source unit. In such cases, the user's presentation can be impeded when the user must carry the extra unit by hand, keep it on the podium or other furniture, etc.

The present invention frees the user from having to wear, carry or keep track of a separate transmitter/power source unit. The present invention combines the laryngophone and the transmitter/power source unit into one streamlined apparatus which is lightweight, durable, flexible, resilient and waterproof.

### BRIEF SUMMARY OF THE INVENTION

The present invention secures a laryngophone (“throat microphone”), transmitter and power source inside a thin sleeve of durable, lightweight, waterproof material so that the present invention can be worn around the user's neck.

### BRIEF DESCRIPTION OF THE SEVERAL VIEWS OF THE DRAWINGS

FIG. 1 is a view of the surface of the present invention that would be in contact with the skin of the user's neck.

FIG. 2 is a view of the outside of the present invention.

FIG. 3 is a view of the present invention worn by a user facing forward.

FIG. 4 is a view of the present invention worn by a user seen from behind.

FIG. 5 is a side view of the present invention worn by a user.

### DETAILED DESCRIPTION OF THE INVENTION

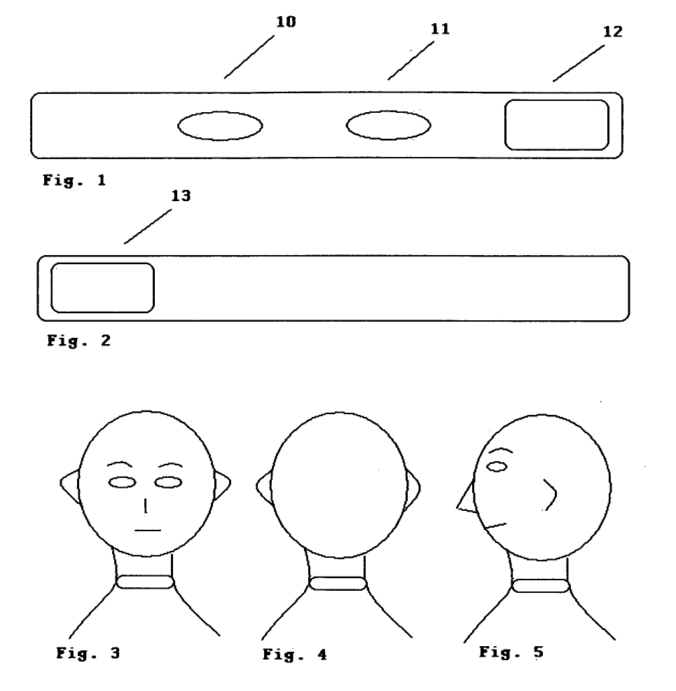

Referring now to the invention in more detail, there is shown in FIG. 1 contact points 10 and 11 which will be against the skin of the user's neck on either side of the user's voice box. Item 12 is a type of affixing device such as a Velcro patch, buckle, snaps, elastic band or other mechanism to secure the invention to user's necks of varying circumferences.

What is claimed is:

1. An apparatus worn around the human neck that contains a laryngophone (“throat microphone”), a transmitter and a power source.

2. The apparatus of claim 1 in which the user's vocalizations are sent from the laryngophone to the transmitter.

3. The apparatus of claim 1 in which the transmitter sends the audio from the laryngophone to a receiver or sound amplification system that is located separately and at some distance from the user.

4. The apparatus of claim 1 in which a power source provides the electricity needed to transmit the audio from the laryngophone to a receiver or sound amplification system.

5. The apparatus of claim 1 which is waterproof, resilient and durable to ensure accurate performance while worn by users engaged in intense physical activity including combat and sports, yet the apparatus of claim 1 is lightweight and flexible so that the user's physical movements and activities are in no way impeded by the apparatus.
